(19) United States
(12) Patent Application Publication
(10) Pub. No.: US 2024/0228748 A1
(43) Pub. Date: Jul. 11, 2024

(54) COMPOSITIONS BASED ON POLYAMIDES AND GLASS FIBERS AND USE THEREOF IN THE MEDICAL FIELD

(71) Applicant: ARKEMA FRANCE, COLOMBES (FR)

(72) Inventors: Amélie NOEL, Serquigny (FR); François RUAS, King Of Prussia, PA (US); Mathieu SABARD, Serquigny (FR)

(73) Assignee: ARKEMA FRANCE, COLOMBES (FR)

(21) Appl. No.: 18/558,856

(22) PCT Filed: Apr. 29, 2022
(86) PCT No.: PCT/FR2022/050828

§ 371 Date: Nov. 3, 2023

(30) Foreign Application Priority Data
May 5, 2021 (FR) FR2104729

(51) Int. Cl. C08K 7/14; A61L 2/07; A61L 2/28; B29C 45/00; B29K 77/00; C08K 5/098; C08K 5/12

(52) U.S. Cl. CPC C08K 7/14; A61L 2/07; A61L 2/28; B29C 45/0005; C08K 5/098; C08K 5/12; A61L 2202/24; B29K 2077/00; B29K 2995/0017; C08K 2201/004

(57) ABSTRACT

The present invention relates to a molding composition comprising, by weight:
a) 20% to 60%, especially from 20% to 50%, in particular 25% to 39.9%, of at least one long-chain aliphatic polyamide having a number of carbon atoms per nitrogen atom of greater than or equal to 9, in particular greater than or equal to 10,

said at least one polyamide having an inherent viscosity in solution of less than or equal to 1.3, in particular less than or equal to 1.25, as determined in accordance with the standard ISO 307:2007 at a temperature of 20° C.,
b) 40% to 75% of glass fibers, especially from 50% to 75%, in particular from 60% to 70%, of glass fibers, and
c) 0% to 5% by weight, preferably 0.1% to 5% by weight, of at least one additive, the sum of the proportions of each constituent of said composition being equal to 100%.

### TECHNICAL FIELD

The present invention relates to molding compositions based on polyamides and glass fibers and to the use thereof in the medical field.

### PRIOR ART

Medical and surgical instruments are conventionally made of metal and must be carefully maintained in order to avoid corrosion, particularly as regards surgical residues adhering and drying on the instruments (incrustations) or the presence of blood, pus, and tissue and bone residues on the instruments after use.

They must also be perfectly sterilized in order to be able to be reused.

Disposable or reusable medical and surgical instruments made of plastic material (or molding composition) are increasingly now appearing on the market.

However, the rigidity of these instruments made of plastic material must be high since said plastic material is a replacement for the metal. The plastic material must therefore exhibit a high modulus and a high stress.

This plastic material must also be easily processable by an injection molding process, in particular with a low mold temperature (for example <100° C.), in order to manufacture these instruments.

The injection-molded parts (or instruments) must exhibit good gloss and also low warping, in particular for thin parts, and they must also withstand sterilization treatments, especially steam sterilization, gamma sterilization, ethylene oxide sterilization and electron beam (ebeam) sterilization, while having low moisture uptake.

In addition, the injection-molded parts must have good chemical resistance to the cleaning products used in hospitals.

It is therefore necessary to find compositions or formulations which, after injection molding, exhibit this compromise of properties. Thus, the present invention relates to a molding composition comprising, by weight:
a) 20% to 60%, especially from 20% to 50%, in particular 25% to 39.9%, of at least one long-chain aliphatic polyamide having a number of carbon atoms per nitrogen atom of greater than or equal to 9, in particular greater than or equal to 10,
said at least one polyamide having an inherent viscosity in solution of less than or equal to 1.3, in particular less than or equal to 1.25, as determined in accordance with the standard ISO 307:2007 at a temperature of 20° C.,
b) 40% to 75% of glass fibers, especially from 50% to 75%, in particular from 60% to 70%, of glass fibers, and
c) 0% to 5% by weight, preferably 0.1% to 5% by weight, of at least one additive,
the sum of the proportions of each constituent of said composition being equal to 100%.

The inventors have thus found that the selection of at least one long-chain aliphatic polyamide having a particular inherent viscosity in solution with a particular range of glass fibers made it possible to constitute a composition which, after injection molding, exhibits the compromise of properties defined above.

The inherent viscosity in solution is determined in accordance with the standard ISO 307:2007 modified in that the solvent is m-cresol rather than sulfuric acid, in that the concentration is 0.5% by weight and in that the temperature is 20° C.

A molding composition according to the invention is a composition transformed, for example, by injection molding, injection blow molding, expansion molding and rotational molding.

### Regarding the Aliphatic Polyamide

The molding composition comprises from 20% to 60%, especially from 20% to 50%, in particular 25% to 39.9%, of at least one long-chain aliphatic polyamide having a number of carbon atoms per nitrogen atom of greater than or equal to 9, in particular greater than or equal to 10.

The nomenclature used to define the polyamides is described in the standard ISO 1874-1:2011, “Plastics—Polyamide (PA) Moulding and Extrusion Materials—Part 1: Designation”, in particular on page 3 (tables 1 and 2), and is well known to a person skilled in the art.

The polyamide can be a homopolyamide or a copolyamide or a mixture thereof.

Said polyamide is advantageously a semicrystalline polyamide or copolyamide.

For the purposes of the invention, the term “semicrystalline” denotes a polyamide or copolyamide which has a melting point (Tm) in DSC according to the standard ISO 11357-3:2013, and an enthalpy of crystallization during the cooling step at a rate of 20 K/min in DSC measured in accordance with the standard ISO 11357-3 of 2013 which is greater than 20 J/g, preferably greater than 30 J/g.

Said polyamide is derived from a repeating unit obtained by polycondensation:
of at least one C₉ to C₁₈, preferentially C₁₀ to C₁₈, more preferentially C₁₀ to C₁₂, amino acid, or
of at least one C₉ to C₁₈, preferentially C₁₀ to C₁₈, more preferentially C₁₀ to C₁₂, lactam, or
of at least one C₄-C₃₆, preferentially C₆-C₁₈, preferentially C₆-C₁₂, more preferentially C₁₀-C₁₂, diamine Ca with at least one C₄-C₃₆, preferentially C₆-C₁₈, preferentially C₆-C₁₂, more preferentially C₁₀-C₁₂, dicarboxylic acid Cb,
or a mixture thereof,
provided that the number of carbon atoms per nitrogen atom in the repeating unit is greater than or equal to 9, in particular greater than or equal to 10.

Advantageously, the long-chain aliphatic polyamide is obtained by polycondensation:
of at least one C₉ to C₁₈, preferentially C₁₀ to C₁₈, especially C₁₀ to C₁₂, amino acid, or
of at least one C₉ to C₁₈, preferentially C₁₀ to C₁₈, especially C₁₀ to C₁₂, lactam.

A C₉ to C₁₂ amino acid is in particular 9-aminononanoic acid, 10-aminodecanoic acid, 10-aminoundecanoic acid, 12-aminododecanoic acid and 11-aminoundecanoic acid and also derivatives thereof, in particular N-heptyl-11-aminoundecanoic acid.

A C₉ to C₁₂ lactam is in particular lauryllactam.

Said at least one C₄-C₃₆ diamine Ca may be chosen in particular from 1,4-butanediamine, 1,5-pentamethylenediamine, 1,6-hexamethylenediamine, 1,7-heptamethylenediamine, 1,8-octamethylenediamine, 1,9-nonamethylenediamine, 1,10-decamethylenediamine, 1,11-undecamethylenediamine, 1,12-dodecamethylenediamine, 1,13-tridecamethylenediamine, 1,14-tetradecamethylenediamine, 1,16-hexadecamethylenediamine and 1,18-octadecamethylenediamine, octadecenediamine, eicosanediamine, docosanediamine and diamines obtained from fatty acids.

Advantageously, said at least one diamine Ca is C₆-C₁₈ and is chosen from 1,6-hexamethylenediamine, 1,7-heptamethylenediamine, 1,8-octamethylenediamine, 1,9-nonamethylenediamine, 1,10-decamethylenediamine, 1,11-undecamethylenediamine, 1,12-dodecamethylenediamine, 1,13-tridecamethylenediamine, 1,14-tetradecamethylenediamine, 1,16-hexadecamethylenediamine and 1,18-octadecamethylenediamine.

Said at least one C₄-C₃₆ dicarboxylic acid Cb may be chosen from succinic acid, glutaric acid, adipic acid, suberic acid, azelaic acid, sebacic acid, undecanedioic acid, dodecanedioic acid, brassylic acid, tetradecanedioic acid, pentadecanedioic acid, hexadecanedioic acid, octadecanedioic acid, octadecenediamine, eicosanediamine, docosanediamine and diamines obtained from fatty acids.

Advantageously, said at least one dicarboxylic acid Cb is C₆-C₁₈ and is chosen from adipic acid, suberic acid, azelaic acid, sebacic acid, undecanedioic acid, dodecanedioic acid, brassylic acid, tetradecanedioic acid, hexadecanedioic acid and octadecanedioic acid.

In particular, the long-chain aliphatic polyamide is chosen from:
polyamide 1010 (PA1010), polyamide 1012 (PA1012), polyamide 1212 (PA1012), polyamide 11 (PA11), polyamide 12 (PA12), or a mixture thereof or a copolyamide thereof.

Advantageously, the polyamide is in particular PA11 and PA12.

In one embodiment, said long-chain aliphatic polyamide is a mixture of two polyamides in a weight ratio range from 5/95 to 95/5.

Advantageously, said mixture is a mixture of two identical polyamides having the same number of carbon atoms per nitrogen atom but with an inherent viscosity in solution of one of the two polyamides of greater than 1.3, but on the condition that the mixture satisfies the condition of having an inherent viscosity in solution of less than or equal to 1.3, in particular less than or equal to 1.25.

### Regarding the Glass Fibers

The glass fibers are present at from 40% to 75%, especially from 50% to 75%, in particular from 60% to 70%, by weight relative to the total weight of the composition.

The glass fibers may be solid and/or hollow; advantageously they are solid.

The glass fibers are advantageously short.

The short glass fibers preferably have a length of between 2 and 13 mm and preferably from 3 to 8 mm before use of the compositions.

Advantageously, the short glass fibers have a fiber length of from 120 to 350 μm.

This fiber length of from 120 to 350 μm is measured on the compound.

The short glass fibers may be of circular or non-circular cross section.

A fiber of circular cross section is defined as a fiber having at any point on its circumference an equal distance to the center of the fiber and therefore represents a perfect or near-perfect circle.

Any glass fiber that does not have this perfect or near-perfect circle is therefore defined as a fiber of non-circular cross section.

Examples of fibers of non-circular cross section, without being limited thereto, are non-circular fibers, having for example an elliptical, oval or cocoon shape, star-shaped fibers, flake-shaped fibers, flat fibers, cruciforms, a polygon and a ring.

The glass fiber may be:
either of circular cross section with a diameter of from 4 μm to 25 μm, preferably from 4 to 15 μm;
or of non-circular cross section with an L/D ratio (L representing the largest dimension of the cross section of the fiber and D the smallest dimension of the cross section of said fiber) of from 2 to 8, in particular from 2 to 4. L and D can be measured by scanning electron microscopy (SEM).

Advantageously, the glass fibers are circular.

### Regarding the Additive

The at least one additive is optionally present at from 0% to 5% by weight, in particular from 0.1% to 5%, relative to the total weight of the composition.

The additive is chosen from fillers, dyes, stabilizers, plasticizers, surface-active agents, nucleating agents, pigments, brighteners, antioxidants, lubricants, flame retardants, natural waxes, additives for laser marking, and mixtures thereof.

By way of example, the stabilizer may be a UV stabilizer, an organic stabilizer or more generally a combination of organic stabilizers, such as an antioxidant of phenol type (for example of the type of that of Irganox® 1010 from Ciba-BASF).

This stabilizer may also be an inorganic stabilizer, such as a copper-based stabilizer. By way of example of such inorganic stabilizers, mention may be made of copper acetates and halides. Incidentally, other metals, such as silver, can optionally be considered, but said metals are known to be less effective. These copper-based compounds are typically combined with halides of alkali metals, in particular potassium.

It would not be a departure from the scope of the invention to use a mixture of plasticizers.

By way of example, the fillers may be chosen from carbon black, talc, pigments and metal oxides (titanium oxide).

### Regarding the Molding Composition

Throughout the description, all the percentages of the composition are given by weight.

The molding composition comprises, by weight:
a) 20% to 60%, especially from 20% to 50%, in particular 25% to 39.9%, of at least one long-chain aliphatic polyamide having a number of carbon atoms per nitrogen atom of greater than or equal to 9, in particular greater than or equal to 10,
said at least one polyamide having an inherent viscosity in solution of less than or equal to 1.3, in particular less than or equal to 1.25, as determined in accordance with the standard ISO 307:2007 at a temperature of 20° C.,
b) 40% to 75% of glass fibers, especially from 50% to 75%, in particular from 60% to 70%, of glass fibers, and
c) 0% to 5% by weight, preferably 0.1% to 5% by weight, of at least one additive,
the sum of the proportions of each constituent of said composition being equal to 100%.

Advantageously, a polyamide other than an aliphatic, in particular semicrystalline, polyamide, is excluded from the composition.

In one embodiment, the molding composition consists of (by weight):
a) 20% to 60%, especially from 20% to 50%, in particular 25% to 39.9%, of at least one long-chain aliphatic polyamide having a number of carbon atoms per nitrogen atom of greater than or equal to 9, in particular greater than or equal to 10,
said at least one polyamide having an inherent viscosity in solution of less than or equal to 1.3, in particular less than or equal to 1.25, as determined in accordance with the standard ISO 307:2007 at a temperature of 20° C.,
b) 40% to 75% of glass fibers, especially from 50% to 75%, in particular from 60% to 70%, of glass fibers, and
c) 0% to 5% by weight, preferably 0.1% to 5% by weight, of at least one additive,
the sum of the proportions of each constituent of said composition being equal to 100%.

In a first variant, the molding composition comprises, by weight:
a) 20% to 50%, in particular 25% to 39.9%, of at least one long-chain aliphatic polyamide having a number of carbon atoms per nitrogen atom of greater than or equal to 9, in particular greater than or equal to 10,
said at least one polyamide having an inherent viscosity in solution of less than or equal to 1.3, in particular less than or equal to 1.25, as determined in accordance with the standard ISO 307:2007 at a temperature of 20° C.,
b) 50% to 75%, in particular from 60% to 70%, of glass fibers, and
c) 0% to 5% by weight, preferably 0.1% to 5% by weight, of at least one additive,
the sum of the proportions of each constituent of said composition being equal to 100%.

In one embodiment of this first variant, the molding composition comprises, by weight:
a) 20% to 49.9%, in particular 25% to 39.9%, of at least one long-chain aliphatic polyamide having a number of carbon atoms per nitrogen atom of greater than or equal to 9, in particular greater than or equal to 10,
said at least one polyamide having an inherent viscosity in solution of less than or equal to 1.3, in particular less than or equal to 1.25, as determined in accordance with the standard ISO 307:2007 at a temperature of 20° C.,
b) 50% to 75%, in particular from 60% to 70%, of glass fibers, and
c) 0.1% to 5% by weight of at least one additive,
the sum of the proportions of each constituent of said composition being equal to 100%.

In another embodiment of this first variant, the composition consists of:
a) 20% to 50%, in particular 25% to 39.9%, of at least one long-chain aliphatic polyamide having a number of carbon atoms per nitrogen atom of greater than or equal to 9, in particular greater than or equal to 10,
said at least one polyamide having an inherent viscosity in solution of less than or equal to 1.3, in particular less than or equal to 1.25, as determined in accordance with the standard ISO 307:2007 at a temperature of 20° ° C.,
b) 50% to 75%, in particular from 60% to 70%, of glass fibers, and
c) 0% to 5% by weight, preferably 0.1% to 5% by weight, of at least one additive,
the sum of the proportions of each constituent of said composition being equal to 100%.

In yet another embodiment of this first variant, the composition consists of:
a) 20% to 49.9%, in particular 25% to 39.9%, of at least one long-chain aliphatic polyamide having a number of carbon atoms per nitrogen atom of greater than or equal to 9, in particular greater than or equal to 10,
said at least one polyamide having an inherent viscosity in solution of less than or equal to 1.3, in particular less than or equal to 1.25, as determined in accordance with the standard ISO 307:2007 at a temperature of 20° C.,
b) 50% to 75%, in particular from 60% to 70%, of glass fibers, and
c) 0.1% to 5% by weight of at least one additive,
the sum of the proportions of each constituent of said composition being equal to 100%.

In a second variant, the molding composition comprises, by weight:
a) 25% to 40%, in particular 25% to 39.9%, of at least one long-chain aliphatic polyamide having a number of carbon atoms per nitrogen atom of greater than or equal to 9, in particular greater than or equal to 10,
said at least one polyamide having an inherent viscosity in solution of less than or equal to 1.3, in particular less than or equal to 1.25, as determined in accordance with the standard ISO 307:2007 at a temperature of 20° C.,
b) 60% to 70% of glass fibers, and
c) 0% to 5% by weight, preferably 0.1% to 5% by weight, of at least one additive,
the sum of the proportions of each constituent of said composition being equal to 100%.

In one embodiment of this second variant, the molding composition comprises, by weight:
a) 25% to 39.9% of at least one long-chain aliphatic polyamide having a number of carbon atoms per nitrogen atom of greater than or equal to 9, in particular greater than or equal to 10,
said at least one polyamide having an inherent viscosity in solution of less than or equal to 1.3, in particular less than or equal to 1.25, as determined in accordance with the standard ISO 307:2007 at a temperature of 20° C.,
b) 60% to 70% of glass fibers, and
c) 0.1% to 5% by weight of at least one additive,
the sum of the proportions of each constituent of said composition being equal to 100%.

In another embodiment of this second variant, the molding composition consists of:
a) 25% to 40%, in particular 25% to 39.9%, of at least one long-chain aliphatic polyamide having a number of carbon atoms per nitrogen atom of greater than or equal to 9, in particular greater than or equal to 10,
said at least one polyamide having an inherent viscosity in solution of less than or equal to 1.3, in particular less than or equal to 1.25, as determined in accordance with the standard ISO 307:2007 at a temperature of 20° ° C.,
b) 60% to 70% of glass fibers, and
c) 0% to 5% by weight, preferably 0.1% to 5% by weight, of at least one additive,
the sum of the proportions of each constituent of said composition being equal to 100%.

In yet another embodiment of this second variant, the molding composition consists of:
a) 25% to 39.9% of at least one long-chain aliphatic polyamide having a number of carbon atoms per nitrogen atom of greater than or equal to 9, in particular greater than or equal to 10,
said at least one polyamide having an inherent viscosity in solution of less than or equal to 1.3, in particular less than or equal to 1.25, as determined in accordance with the standard ISO 307:2007 at a temperature of 20° C.,
b) 60% to 70% of glass fibers, and
c) 0.1% to 5% by weight of at least one additive,
the sum of the proportions of each constituent of said composition being equal to 100%.

According to another aspect, the present invention relates to the use of a composition as defined above for the manufacture of an article for the medical field, in particular for single or multiple use.

All of the embodiments described above are valid for this “use” paragraph.

The expression “article for the medical field” denotes, for example, a retractor, scissors, tongs, surgical pliers, tweezer forceps, a hook, a needle holder, a stylet, forceps, a cannula, a trocar, a syringe, a bistoury, an extractor, a spoon, a curette, an ampoule, a catheter, a surgical stapler, a gynecological instrument (such as a speculum), a clamp, handles of surgical instruments, a suction tube, a dilator, a sterilization tray, a cap and any other medical article traditionally made from metal.

The expression “single-use article for the medical field” denotes an article which is used only once and discarded after use, such as a retractor, scissors, tongs, surgical pliers, tweezer forceps, a hook, a needle holder, a stylet, forceps, a cannula, a trocar, a syringe, a bistoury, an extractor, a spoon, a curette, an ampoule, a catheter, a gynecological instrument (such as a speculum), a clamp, handles of surgical instruments, a suction tube, a dilator, a cap and any other single-use medical article traditionally made from metal.

The expression “multiple-use article for the medical field” denotes an article which is reused after use, and which requires cleaning and/or sterilization in order to be able to be reused.

Sterilization can be effected by steam or by gamma rays.

In one embodiment, said article is a surgical instrument.

Advantageously, the article after steam sterilization exhibits a variation in cross section of less than 3% compared to the cross section before sterilization, determined on a type 1A test specimen of 4×10 mm² cross section.

In one embodiment, the article exhibits very good resistance to gamma-ray sterilization.

In another embodiment, the article after steam sterilization exhibits a variation in stress of less than 20% compared to the breaking stress before sterilization, determined in accordance with the standard ISO 527:2012 on a type 1A test specimen.

Advantageously, the article is manufactured by injection molding.

According to yet another aspect, the present invention relates to an article obtained by injection molding with a composition as defined above.

### EXAMPLES

Preparation of the compositions of the invention and mechanical properties:

The compositions of table 1 were prepared by melt mixing polyamide pellets with glass fibers and optionally additives.

The PA11- and PA12-based compositions were processed by compounding on a co-rotating twin-screw extruder with a diameter of 40 mm with a flat temperature profile)(T° at 250° C. The screw speed is 300 rpm and the throughput is 100 kg/h. As for the PA66-based compositions, these were processed with a flat temperature profile at 300° C. The other parameters remain unchanged.

The glass fibers are introduced via side feeding.

The semicrystalline aliphatic polyamide and optionally the additives are added via the main hopper.

The compositions were then molded on an (Engel brand) injection molding machine at a setpoint temperature of 260° C. (for the PA11- and PA12-based compositions) and a mold temperature of 70° C. in the form of dumbbells or bars in order to study the properties of the compositions according to the standards below. The PA66-based compositions were injected at a temperature of 300° C. with a mold temperature of 110° C.

The tensile modulus was measured at 23° C. in accordance with the standard ISO 527-1:2012 on type 1A dumbbells, before sterilization (i.e. at t0, on dry samples), after steam sterilization or after gamma sterilization.

The elongation and the stress at break were also measured at 23° C. in accordance with this same standard ISO 527-1:2012 before sterilization, after steam sterilization or after gamma sterilization.

The crosshead speed was fixed at 1 mm/min for the measurement of the modulus and 5 mm/min for the measurement of the elongation and the stress. An Instron 5966 type machine is used.

The test conditions are 23° C.+/−2° C., on dry samples (before sterilization step, i.e. at t0) and on conditioned samples (i.e. after the sterilization step).

The steam sterilization of the test specimens is carried out as follows: the 1A dumbbells underwent 25 sterilization cycles, at 134° C., 2 bar, for 12 minutes. Between each cycle, the sample is maintained at 50° C. for 5 minutes.

On conclusion of the 25 steam sterilization cycles, a tensile test was performed for each composition, in accordance with the standard ISO 527-1:2012.

The gloss was determined visually. Two appearance levels were assigned: glossy or matte appearance.

The impact strength was determined in accordance with ISO 179-1:2010/1eA (Charpy impact) on bars of dimensions 80 mm×10 mm×4 mm, V-shaped notch, dry, at a temperature of 23° C.+/−2° C. under a relative humidity of 50%+/−10%.

The “gamma” sterilization consists in applying an ionizing dose of 50 000 gray (using gamma radiation (from cobalt 60)) to 1A dumbbells (for each composition). On conclusion of this sterilization, a tensile test was performed for each composition, in accordance with the standard ISO 527-1:2012. Plates of 100*100*1 mm3 were also prepared by injection molding the various compositions:
Injection temperature (feed/nozzle): 270/290° C.
Mold temperature: See table 1
Hold time: 10 seconds
Cooling time: 20 seconds
   and the warping was evaluated, after 7 days, according to the method described below:

The 1 mm thick plates of dimensions 100 mm×100 mm are placed on a flat support; the operator presses on 3 corners of the plate in order to cause the fourth to raise. The distance between the surface of the table and the sample is then measured. The warping is then calculated by the following formula: warping (%)=(distance between the support and the plate (in mm)/100 mm×100).

TABLE 1

| | E1 | E2 | E3 | E4 | E5 | CE1 | CE2 | CE3 | CE4 |
|---|---|---|---|---|---|---|---|---|---|
| PA11-iv m-cresol = 1.18 | 34 | 34 | | 49 | | | | | |
| PA11-iv m-cresol = 1.41 | | | | | | 34 | | | |
| PA11-iv m-cresol = 1.27 | | | | | 34 | | | | |
| PA11-iv m-cresol = 1.33 | | | | | | | | | 34 |
| PA12-Iv m-cresol = 1.22 | | | 34 | | | | | | |
| PA66-iv m cresol = 1.12 | | | | | | | 49 | 34 | |
| CSG-3PA820 glass fiber | | 65 | | | | | | | |
| FoodContact ™ 295-10 | 65 | | 65 | 50 | 65 | 65 | 50 | 65 | 65 |
| glass fiber | | | | | | | | | |
| Licowax E | 0.3 | 0.3 | 0.3 | 0.3 | 0.3 | 0.3 | 0.3 | 0.3 | 0.3 |
| Calcium stearate | 0.3 | 0.3 | 0.3 | 0.3 | 0.3 | 0.3 | 0.3 | 0.3 | 0.3 |
| Irganox 1010 | 0.4 | 0.4 | 0.4 | 0.4 | 0.4 | 0.4 | 0.4 | 0.4 | 0.4 |

E = composition according to the invention
CE = counterexample

The FoodContact™ 295-10 glass fiber (of circular cross section with 10 μm diameter) is sold by the company Owens Corning.

The CSG3PA820 glass fiber (of non-circular cross section with 7×28 μm cross section) is sold by the company Nitto Boseki.

The PA11 and PA12 are produced by the applicant company.

The calcium stearate is sold by the company Greven.

Licowax E is sold by the company Clariant.

Irganox 1010 is sold by BASF.

The PA66 (reference Stabamid 25AE1) is sold by Domo Chemicals.

TABLE 2

| | E1 | E2 | E3 | E4 | E5 | CE1 | CE2 | CE3 | CE4 |
|---|---|---|---|---|---|---|---|---|---|
| Tensile modulus (GPa)-t0-dry | 19 | 19.2 | 18.6 | 13.2 | 18.4 | 18.7 | 19.2 | 24.1 | 18.2 |
| state-23° C. | | | | | | | | | |
| Breaking stress (MPa)-t0-dry | 190 | 195 | 187 | 178 | 187 | 185 | 255 | 265 | 185 |
| state-23° C. | | | | | | | | | |
| Elongation at break (%)-t0- | 3.4 | 3.2 | 3.2 | 5.5 | 3.6 | 3.5 | 2.6 | 2.0 | 3.8 |
| dry state-23° C. | | | | | | | | | |
| Tensile modulus (GPa)-after 25 | 14.6 | 14.9 | 14.3 | 10.5 | 14.4 | 14.2 | 11.4 | 13.5 | 14.1 |
| cycles of steam sterilization- | | | | | | | | | |
| 23° C. | | | | | | | | | |
| Breaking stress (MPa)-after 25 | 163 | 165 | 161 | 153 | 161 | 158 | 136 | 143 | 158 |
| cycles of steam sterilization- | | | | | | | | | |
| 23° C. | | | | | | | | | |
| Variation in stress (between | −14% | −15% | −14% | −14% | −14% | −15% | −47% | −46% | −15% |
| state t0 and after sterilization)- | | | | | | | | | |
| % | | | | | | | | | |
| Elongation at break (%)-after | 3.9 | 3.1 | 3.7 | 5.8 | 4.1 | 4.1 | 2.7 | 2.2 | 4.2 |
| 25 cycles of steam sterilization- | | | | | | | | | |
| 23° C. | | | | | | | | | |
| Variation in cross section (t0 vs. | 0.6% | 0.5% | 0.7% | 0.8% | 0.6% | 0.7% | 3.4% | 3.3% | 0.7% |
| after 25 cycles of steam | | | | | | | | | |
| sterilization) | | | | | | | | | |
| Tensile modulus (GPa)-after | 18.7 | 18.8 | 18.2 | 13.1 | 18.3 | 18.5 | 18.8 | 23.9 | 18.1 |
| gamma sterilization (under 50 | | | | | | | | | |
| kGy)-23° C. | | | | | | | | | |
| Breaking stress (MPa)-after | 186 | 194 | 183 | 151 | 183 | 179 | 251 | 261 | 179 |
| gamma sterilization-23° C. | | | | | | | | | |
| Elongation at break (%)-after | 3.3 | 3.1 | 3.3 | 5.6 | 3.5 | 3.4 | 2.4 | 2.1 | 3.7 |
| gamma sterilization-23° C. | | | | | | | | | |
| Mold temperature at injection | 70° C. | 70° C. | 70° C. | 70° C. | 70° C. | 70° C. | 110° C. | 110° C. | 70° C. |
| Gloss | Glossy | Glossy | Glossy | Glossy | Glossy | Matte | Glossy | Glossy | Matte |
| Processability by an injection | Good | Good | Good | Good | Good | Not | Not | Not | Average |
| molding process | | | | | | good | good | good | |
| Resilience (kJ/m²)-23° C. | 19 | 21 | 18 | 19 | 20 | 20 | 12 | 8 | 21 |
| (ISO179-1eA)-dry state | | | | | | | | | |
| Warping on 100 × 100 × 1 mm | No | No | No | No | Low | Warp- | No | Warp- | Warp- |
| plates, after injection molding | warp- | warp- | warp- | warp- | warp- | ing | warp- | ing | ing |
| | ing, | ing, | ing, | ing, | ing | >2% | ing | >2% | >2% |
| | <1% | <1% | <1% | <1% | <1% | | <1% | | |

1. A molding composition comprising, by weight:
a) 20% to 60% of at least one long-chain aliphatic polyamide having a number of carbon atoms per nitrogen atom of greater than or equal to 9, said at least one polyamide having an inherent viscosity in solution of less than or equal to 1.3, as determined in accordance with the standard ISO 307:2007 at a temperature of 20° C.,
b) 40% to 75% of glass fibers, said glass fibers being short glass fibers of circular cross section and having a fiber length of from 120 to 350 μm, and
c) 0% to 5% by weight of at least one additive, the sum of the proportions of each constituent of said composition being equal to 100%.

2. The composition as claimed in claim 1, wherein at least two long-chain aliphatic polyamides are present in the composition.

3. The composition as claimed in claim 1, wherein the long-chain aliphatic polyamide is obtained by polycondensation:
of at least one C₉ to C₁₈ amino acid, or of at least one C₉ to C₁₈ lactam, or of at least one aliphatic diamine Ca of at least one C₄-C₃₆ diamine Ca with at least one C₄-C₃₆ dicarboxylic acid Cb, or a mixture thereof.

4. The composition as claimed in claim 1 wherein the long-chain aliphatic polyamide is obtained by polycondensation:
of at least one C₉ to C₁₈ amino acid, or of at least one C₉ to C₁₈ lactam.

5. The composition as claimed in claim 1, wherein said long-chain polyamide is chosen from PA1010, PA1012, PA1212, PA11 and PA 12.

6. The composition as claimed in claim 1, wherein said long-chain polyamide is chosen from PA11 and PA 12.

7. The composition as claimed in claim 1, wherein said at least one additive is chosen from fillers, dyes, stabilizers, plasticizers, surface-active agents, nucleating agents, pigments, brighteners, antioxidants, lubricants, flame retardants, natural waxes, additives for laser marking, and mixtures thereof.

8. A method for the manufacture of an article for the medical field, the method comprising manufacturing the article from the composition as defined in claim 1.

9. The method as claimed in claim 8, wherein the article for the medical field is a surgical instrument.

10. The method as claimed in claim 9, wherein the article after steam sterilization exhibits a variation in cross section of less than 3% compared to the cross section before sterilization, determined on a type 1A test specimen.

11. The method as claimed in claim 9, wherein the article exhibits very good resistance to gamma-ray sterilization.

12. The method as claimed in claim 10, wherein the article after steam sterilization exhibits a variation in stress of less than 20% compared to the breaking stress before sterilization, determined in accordance with the standard ISO 527:2012 on a type 1A test specimen.

13. The method in claim 8, wherein the article is manufactured by injection molding.

14. An article obtained by injection molding with a composition as defined in claim 1.
